(19) United States
(12) Patent Application Publication
(10) Pub. No.: US 2018/0222922 A1
(43) Pub. Date: Aug. 9, 2018

(54) RADIOFLUORINATED 7-AMINO-5-THIO-THIAZOLO[4,5-D]PYRIMIDINES FOR IMAGING FRACTALKINE RECEPTOR (CX3CR1)

(71) Applicant: THE JOHNS HOPKINS UNIVERSITY, BALTIMORE, MD (US)

(72) Inventors: MARTIN G. POMPER, BALTIMORE, MD (US); RONNIE C. MEASE, FAIRFAX, VA (US); XING YANG, BALTIMORE, MD (US); CATHERINE FOSS, BALTIMORE, MD (US)

(73) Assignee: THE JOHNS HOPKINS UNIVERSITY, BALTIMORE, MD (US)

(21) Appl. No.: 15/580,779

(22) PCT Filed: Jun. 8, 2016
(86) PCT No.: PCT/US16/36447

§ 371 Date: Dec. 8, 2017

Related U.S. Application Data
(60) Provisional application No. 62/172,547, filed on Jun. 8, 2015.

(51) Int. Cl. C07D 513/04; A61K 51/04

(52) U.S. Cl. CPC C07D 513/04; A61K 51/0459

(57) ABSTRACT

Radiofluorinated 7-Amino-5-thio-thiazolo[4,5-d]pyrimidines targeting Fractalkine Receptor (CX₃CR1) are disclosed. Methods of imaging CX₃CR1-expressing tumors or cells also are disclosed.

### CROSS-REFERENCE TO RELATED APPLICATIONS

This application claims the benefit of U.S. Provisional Application No. 62/172,547, filed Jun. 8, 2015, which is incorporated herein by reference in its entirety.

### BACKGROUND

Microglia constitute 10-15% of the total glial cell population within the brain. They constantly scavenge the central nervous system (CNS) for plaques, damaged neurons and infectious agents. Microglia are extremely sensitive to even small pathological changes in the CNS. They play important role in neuroinflammation, neurodegeneration and infection diseases. Many groups worldwide have endeavored to detect and quantify microglial activation with imaging to understand a variety of neuropsychiatric diseases, ranging from HIV dementia to schizophrenia—all of which have a prominent neuroinflammatory component. The emerging literature indicates that fractalkine receptor (CX₃CR1) is important in microglial pro-inflammatory biology (Hellwig et al., 2013). It also is the only known microglia-specific target indicated in neuroinflammation within the CNS, and happens to be a readily accessible receptor. In the periphery, CX₃CR1 also plays important roles in inflammatory diseases, cell migration and adhesion. Therefore, being able to image activated microglia is of great clinical importance.

Currently, there are no non-invasive techniques to specifically study brain inflammation, although a number of targets are under consideration. Because CX₃CR1 is specific to microglia within the CNS and is located on the plasma membrane, it is an excellent target to study that process. Within the last year, low-molecular-weight (LMW) agents that target CX₃CR1 have been reported (Karlstrom et al., 2013). However, to date, no positron emission tomography (PET) agents, especially F-18 labeled agents, are routinely available to study CX₃CR1. Therefore, a need remains for CX₃CR1 specific low-molecular-weight (LMW) radiotracers as they would have a broad utility, especially for brain imaging, and provide unprecedented and specific information for the detection and treatment of inflammatory diseases.

### SUMMARY

In some aspects, the presently subject matter provides compounds of formula (Ia) or (Ib):

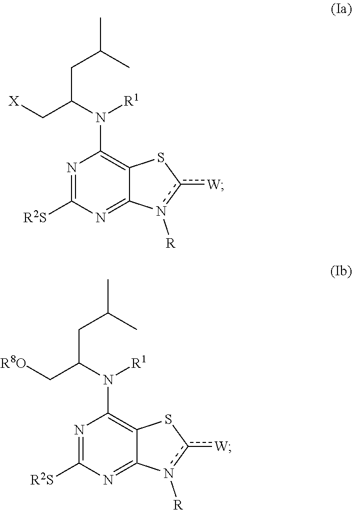

wherein: R can be present or absent and is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl; R¹ is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl; R² is selected from the group consisting of of hydrogen, substituted or unsubstituted alkyl, substituted or unsubstituted aryl, substituted or unsubstituted heteroalkyl, substituted or unsubstituted heteroaryl, substituted or unsubstituted arylalkyl, substituted or unsubstituted alkylaryl, substituted or unsubstituted naphthyl, and substituted or unsubstituted biphenyl, and

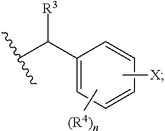

R³ is selected from the group consisting of hydrogen, amine, hydroxyl, carboxyl, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, and substituted or unsubstituted heteroalkylaryl, substituted or unsubstituted naphthyl, and substituted or unsubstituted biphenyl; R⁴ is selected from the group consisting of halogen, alkoxyl, alkyl, alkenyl, alkynyl, aryl, alkylaryl, arylalkyl, —CN, —CF₃, —CONR⁵R⁶, —SO₂R⁷; each R⁵ and R⁶ is independently selected from the group consisting of hydrogen, substituted or unsubstituted alkyl, substituted and unsubstituted aryl, and substituted and unsubstituted heteroaryl; R⁷ is selected from the group consisting of hydrogen, alkyl, hydroxyl, —NR⁵R⁶; R⁸ is selected from the group consisting of hydrogen and a sulfonyl group; n is an integer selected from the group consisting of 0, 1, 2, 3, and 4; X is —NR⁵R⁶ or is selected from the group consisting of F, Br, and I, and radioisotopes thereof; [symbol] represents a single or a double bond; W is selected from the group consisting of ═O, and —NR⁵R⁶; and stereoisomers or pharmaceutically acceptable salt thereof.

In certain aspects, the compound of formula (Ia) or (Ib) further comprises a radioactive isotope suitable for imaging.

In other aspects, the presently disclosed subject matter provides a method for imaging on one or more fractalkine receptors (CX₃CR1)-expressing tumors or cells, the method comprising contacting the one or more tumors or cells with an effective amount of a compound of formula (Ia) or (Ib), and making an image.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1A and FIG. 1B show the imaging study of 2-[18F]FBTTP in healthy mice at a 1-hour time point; (A) PET image; (B) PET image merged with CT; and

FIG. 2 shows general structures of fluorinated 7-amino-5-thio-thiazolo[4,5-d]pyrimidines as PET imaging agents for CX₃CR1.

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Radiofluorinated 7-Amino-5-Thio-Thiazolo[4,5-D]Pyrimidines for Imaging Fractalkine Receptor (CX₃CR1)

A. Compounds of Formula (I)

In some embodiments, the presently disclosed subject matter provides a compound of formula (Ia) or (Ib):

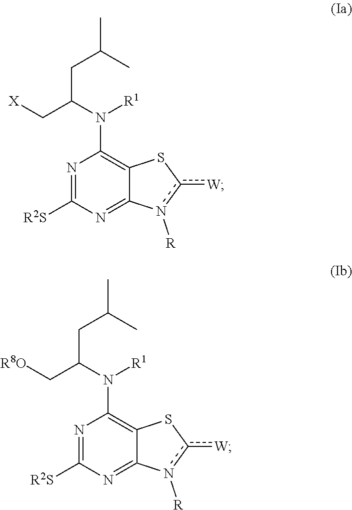

wherein:

wherein: R can be present or absent and is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl; R¹ is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl; R² is selected from the group consisting of of hydrogen, substituted or unsubstituted alkyl, substituted or unsubstituted aryl, substituted or unsubstituted heteroalkyl, substituted or unsubstituted heteroaryl, substituted or unsubstituted arylalkyl, substituted or unsubstituted alkylaryl, substituted or unsubstituted naphthyl, and substituted or unsubstituted biphenyl, and

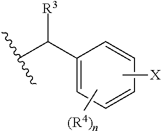

R³ is selected from the group consisting of hydrogen, amine, hydroxyl, carboxyl, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, and substituted or unsubstituted heteroalkylaryl, substituted or unsubstituted naphthyl, and substituted or unsubstituted biphenyl; R⁴ is selected from the group consisting of halogen, alkoxyl, alkyl, alkenyl, alkynyl, aryl, alkylaryl, arylalkyl, —CN, —CF₃, —CONR⁵R⁶, —SO₂R⁷; each R⁵ and R⁶ is independently selected from the group consisting of hydrogen, substituted or unsubstituted alkyl, substituted and unsubstituted aryl, and substituted and unsubstituted heteroaryl; R⁷ is selected from the group consisting of hydrogen, alkyl, hydroxyl, —NR⁵R⁶; R⁸ is selected from the group consisting of hydrogen and a sulfonyl group; n is an integer selected from the group consisting of 0, 1, 2, 3, and 4; X is —NR₅R₆ or is selected from the group consisting of F, Br, and I, and radioisotopes thereof; [symbol] represents a single or a double bond; W is selected from the group consisting of ═O, and —NR⁵R⁶; and stereoisomers or pharmaceutically acceptable salt thereof.

In particular embodiments, the compound of formula (Ia) or (Ib) is selected from the group consisting of:

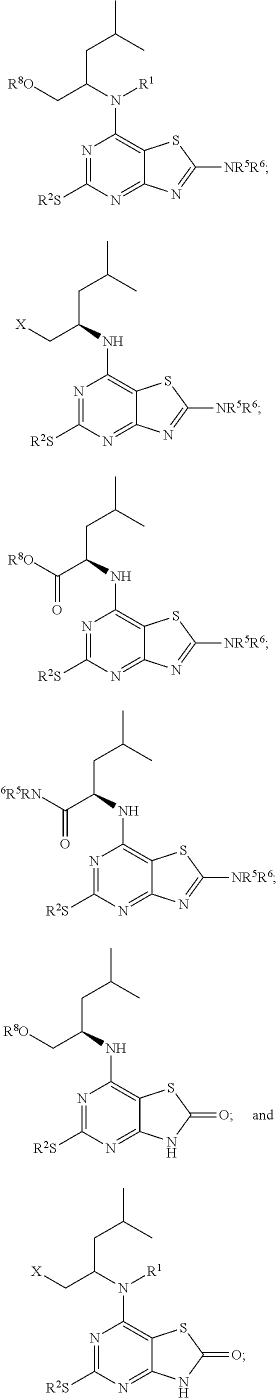

and stereoisomers or pharmaceutically acceptable salt thereof.

In more particular embodiments, the compound of formula (Ib) is:

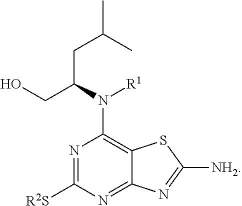

In yet more particular embodiments, the compound of formula (Ib) is selected from the group consisting of:

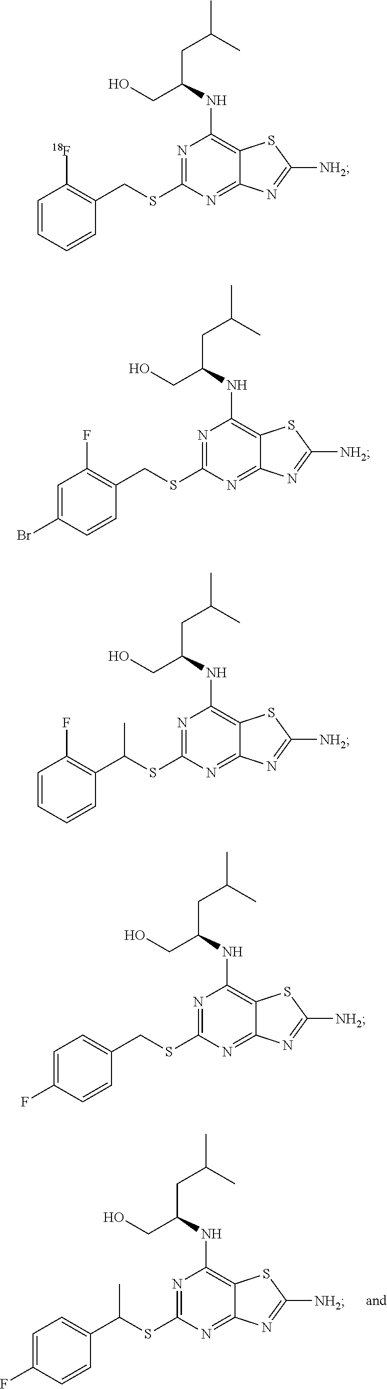 [SMILES: CC(C)C[C@H](CO)Nc1nc(SCc2ccccc2[18F])nc2nc(N)sc12 ; CC(C)C[C@H](CO)Nc1nc(SCc2ccc(Br)cc2F)nc2nc(N)sc12 ; CC(C)C[C@H](CO)Nc1nc(SC(C)c2ccccc2F)nc2nc(N)sc12 ; CC(C)C[C@H](CO)Nc1nc(SCc2ccc(F)cc2)nc2nc(N)sc12 ; CC(C)C[C@H](CO)Nc1nc(SC(C)c2ccc(F)cc2)nc2nc(N)sc12]

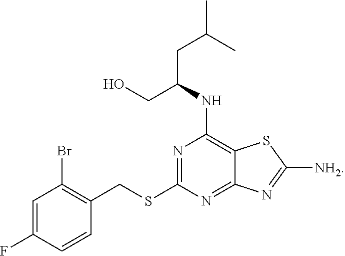 [SMILES: CC(C)C[C@H](CO)Nc1nc(SCc2ccc(F)cc2Br)nc2nc(N)sc12]

In other embodiments, the compound of formula (Ia) or (Ib) comprises a radioactive isotope suitable for imaging. In further embodiments, the radioactive isotope suitable for imaging is selected from the group consisting of ¹⁸F.

B. Methods of Using Compounds of Formula (I) for Imaging CX₃CR1-Expressing Tumors or Cells

In some embodiments, the presently disclosed subject matter provides a method for imaging one or more CX₃CR1-expressing tumors or cells, the method comprising contacting the one or more tumors or cells with an effective amount of a compound of formula (Ia) or (Ib), and making an image, wherein the compound of formula (I) comprises:

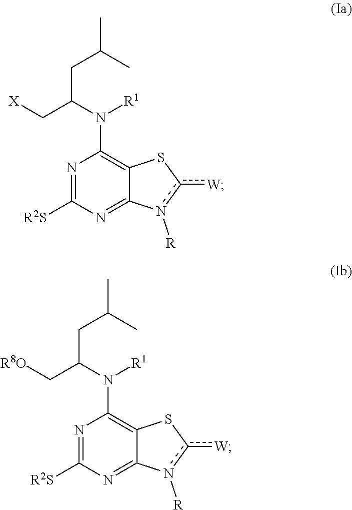

wherein: R can be present or absent and is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl; R¹ is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl; R² is selected from the group consisting of of hydrogen, substituted or unsubstituted alkyl, substituted or unsubstituted aryl, substituted or unsubstituted heteroalkyl, substituted or unsubstituted heteroaryl, substituted or unsubstituted arylalkyl, substituted or unsubstituted alkylaryl, substituted or unsubstituted naphthyl, and substituted or unsubstituted biphenyl, and

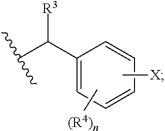

R³ is selected from the group consisting of hydrogen, amine, hydroxyl, carboxyl, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, and substituted or unsubstituted heteroalkylaryl, substituted or unsubstituted naphthyl, and substituted or unsubstituted biphenyl; R⁴ is selected from the group consisting of halogen, alkoxyl, alkyl, alkenyl, alkynyl, aryl, alkylaryl, arylalkyl, —CN, —CF₃, —CONR⁵R⁶, —SO₂R⁷; R⁵ and R⁶ are each independently selected from the group consisting of hydrogen, substituted or unsubstituted alkyl, substituted and unsubstituted aryl, and substituted and unsubstituted heteroaryl; R⁷ is selected from the group consisting of hydrogen, alkyl, hydroxyl, —NR⁵R⁶; R⁸ is selected from the group consisting of H, and a sulfonyl group; n is an integer selected from the group consisting of 0, 1, 2, 3, and 4; X is ¹⁸F; [symbol] represents a single or a double bond; W is selected from the group consisting of ═O, and —NR⁵R⁶; and radioactive isotope suitable for imaging; and stereoisomers or pharmaceutically acceptable salt thereof.

“Contacting” means any action which results in at least one compound comprising the imaging agent of the presently disclosed subject matter physically contacting at least one or more CX₃CR1-expressing tumors or cells. Contacting can include exposing the CX₃CR1-expressing tumors or cells to the compound in an amount sufficient to result in contact of at least one compound with at least one CX₃CR1-expressing tumor or cell. The method can be practiced in vitro or ex vivo by introducing, and preferably mixing, the compound and CX₃CR1-expressing tumors or cells in a controlled environment, such as a culture dish or tube. The method can be practiced in vivo, in which case contacting means exposing at least one CX₃CR1-expressing tumor or cell in a subject to at least one compound of the presently disclosed subject matter, such as administering the compound to a subject via any suitable route. According to the presently disclosed subject matter, contacting may comprise introducing, exposing, and the like, the compound at a site distant to CX₃CR1-expressing tumors or cells to be contacted, and allowing the bodily functions of the subject, or natural (e.g., diffusion) or man-induced (e.g., swirling) movements of fluids to result in contact of the compound and CX₃CR1-expressing tumors or cells.

By “making an image,” it is meant using positron emission tomography to form an image of a cell, tissue, tumor, part of body, and the like.

In particular embodiments, the compound of formula (Ia) or (Ib) is selected from the group consisting of:

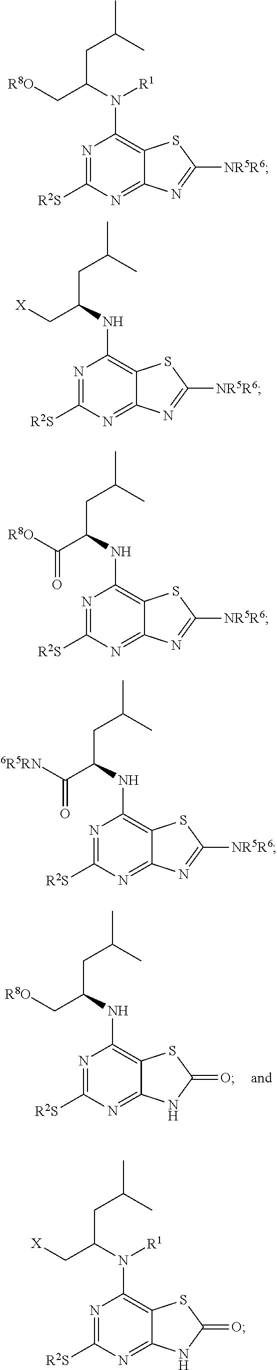

and stereoisomers or pharmaceutically acceptable salt thereof.

In more particular embodiments, the compound of formula (Ib) is:

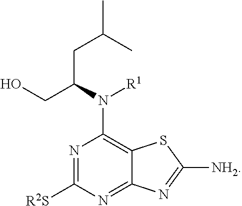

In yet more particular embodiments, the compound of formula (Ib) is selected from the group consisting of:

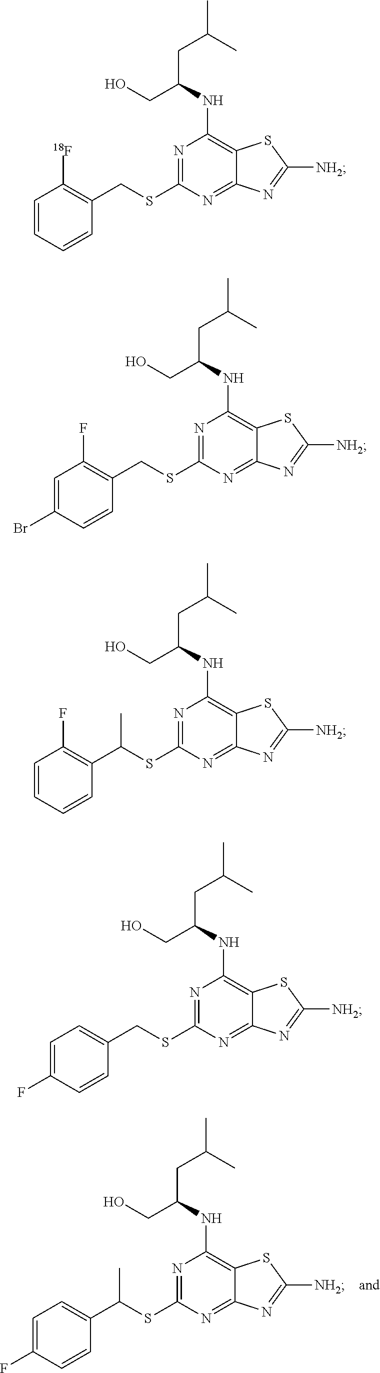 [SMILES: CC(C)C[C@H](CO)Nc1nc(SCc2ccccc2[18F])nc2nc(N)sc12 ; CC(C)C[C@H](CO)Nc1nc(SCc2ccc(Br)cc2F)nc2nc(N)sc12 ; CC(C)C[C@H](CO)Nc1nc(SC(C)c2ccccc2F)nc2nc(N)sc12 ; CC(C)C[C@H](CO)Nc1nc(SCc2ccc(F)cc2)nc2nc(N)sc12 ; CC(C)C[C@H](CO)Nc1nc(SC(C)c2ccc(F)cc2)nc2nc(N)sc12 ; "and"]

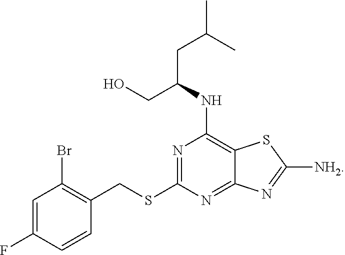 [SMILES: CC(C)C[C@H](CO)Nc1nc(SCc2ccc(F)cc2Br)nc2nc(N)sc12]

In other embodiments, the one or more fractalkine receptors is in vitro, in vivo, or ex vivo.

In certain embodiments, the one or more CX₃CR1-expressing tumors or cells are present in a subject. In some embodiments, the method is non-invasive.

The term “non-invasive” as used herein refers to methods where no instruments are introduced into the body.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject. In some embodiments, a detectably effective amount of the imaging agent of the presently disclosed methods is administered to a subject.

In accordance with the presently disclosed subject matter, “a detectably effective amount” of the imaging agent is defined as an amount sufficient to yield an acceptable image using equipment which is available for clinical use. A detectably effective amount of the imaging agent may be administered in more than one injection. The detectably effective amount of the imaging agent can vary according to factors such as the degree of susceptibility of the individual, the age, sex, and weight of the individual, idiosyncratic responses of the individual, the dosimetry, and instrument and film-related factors. Optimization of such factors is well within the level of skill in the art.

It is preferable to have the compound comprising the imaging agent to localize to the tumor or cell quickly after administration so as to minimize any side effects to the subject. Accordingly, in some embodiments, the compound comprising the imaging agent substantially localizes to the tumor or cell within about 60 minutes of administration.

In some embodiments, the presently disclosed methods use compounds that are stable in vivo such that substantially all, e.g., more than about 50%, 60%, 70%, 80%, or more preferably 90% of the injected compound is not metabolized by the body prior to excretion. In other embodiments, the compound comprising the imaging agent is stable in vivo.

It also is preferable that the compounds of the presently disclosed subject matter are excreted from tissues of the body quickly to prevent prolonged exposure to the radiation of the radiolabeled compound administered to the patient. Typically compounds of the presently disclosed subject matter are eliminated from the body in less than about 24 hours. More preferably, compounds of the presently disclosed subject matter are eliminated from the body in less than about 16 hours, 12 hours, 8 hours, 6 hours, 4 hours, 2 hours, 90 minutes, or 60 minutes.

In general, the “effective amount” of an active agent refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of Formula (Ia) or (Ib) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

The descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, acylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(l-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

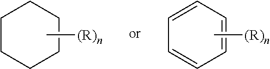

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

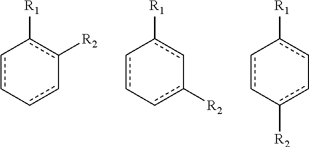

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R″′, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″ ″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituents R, R′, R″ and R″′ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R″′ are each independently selected from the group consisting of alkyl groups. Additionally, R′, R″, and/or R″′ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L-for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner.

The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Synthesis and Use of 2-[¹⁸F]FBTTP and Related Agents for CX₃CR1-Based Target PET Imaging

### Overview

Many neuropsychiatric diseases possess a neuroinflammatory component (Hellwig et al., 2013). Within the central nervous system, the fractalkine receptor (CX₃CR1) is a microglia-specific target and could serve as a new biomarker for imaging neuroinflammation. It is located on the cell surface thereby making it accessible to administered targeting radiotracers. Recently, (R)-2-((2-amino-5-((2-fluorobenzyl)thio)thiazolo[4,5-d]pyrimidin-7-yl)amino)-4-methylpentan-1-ol (2-FBTTP) was one among a library of CX₃CR1 selective antagonists reported (Karlstrom et al., 2013). 2-FBTTP has a Ki of 23 nM and an 18-fold selectivity over CXCR2. In the presently disclosed subject matter, the radiosynthesis of 2-[¹⁸F]FBTTP has been reported, as well as its initial in-vivo evaluation.

(R)-2-((2-amino-5-mercaptothiazolo[4,5-d]pyrimidin-7-yl)amino)-4-methylpentan-1-ol was synthesized as previously reported (Karlstrom et al., 2013) and alkylated with 2-[¹⁸F]fluoro-benzylbromide to produce 2-[¹⁸F]FBTTP. 2-[¹⁸F]fluorobenzylbromide was prepared by a one-pot microwave radiofluorination of 2-formyl-N,N,N-trimethylbenzenanilinium triflate followed by sodium borohydride reduction, and bromination with 48% HBr using a procedure analogous to that reported for the preparation of 4-[¹⁸F]fluoro-benzylbromide (Cho et al., 2012; Ravert et al., 2014). 2-[¹⁸F]FBTTP (350 μCi) was injected via the tail vein of healthy mice and imaged using small animal PET. Images were collected at 10, 30, 60, and 120 min post-injection. 2-[¹⁸F]FBTTP was prepared in a decay corrected yield of 10.2% in a specific activity of 1500 Ci/mmol (55,500 GBq/mmol) in a total synthesis time of 110 min.

### Material and Methods

### Synthesis of (2R)-2-[(2-Amino-5-sulfanyl[1,3]thiazolo[4,5-d]pyrimidin-7-yl)-amino]-4-methylpentan-1-ol 8

The synthesis of (2R)-2-[(2-Amino-5-sulfanyl[1,3]thiazolo[4,5-d]pyrimidin-7-yl)-amino]-4-methylpentan-1-ol 8 which serves as the precursor for the synthesis of 9a-f was achieved in gram scale from compound 1 as previously described (Karlstrom et al., 2013) and as disclosed on Scheme 1.

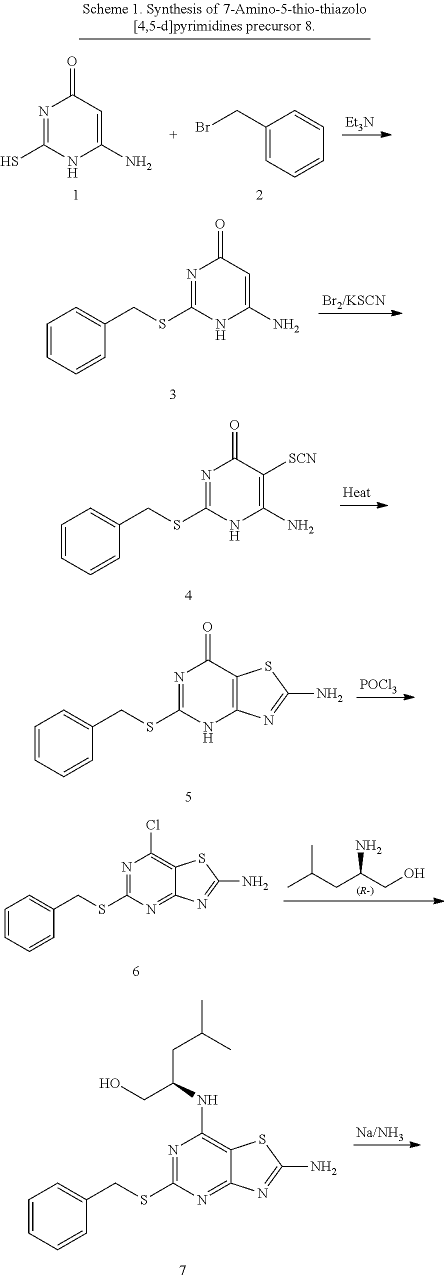

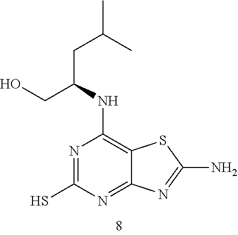 [SMILES: CC(C)C[C@H](CO)Nc1nc(S)nc2nc(N)sc12]

Starting from 8, a series of fluorinated CX₃CR1 ligands 9a-f were prepared as shown in Scheme 2. Among these compounds, the binding affinity of 9d was reported as Ki=23 nM for CX₃CR1 (220 nM for CXCR2), 9e was reported as Ki=4.7 nM for CX₃CR1 (1,400 nM for CXCR2) and 9f was reported as Ki=8.1 nM for CX₃CR1 (1400 nM for CXCR2) which demonstrates the specificity of these ligands for CX₃CR1.

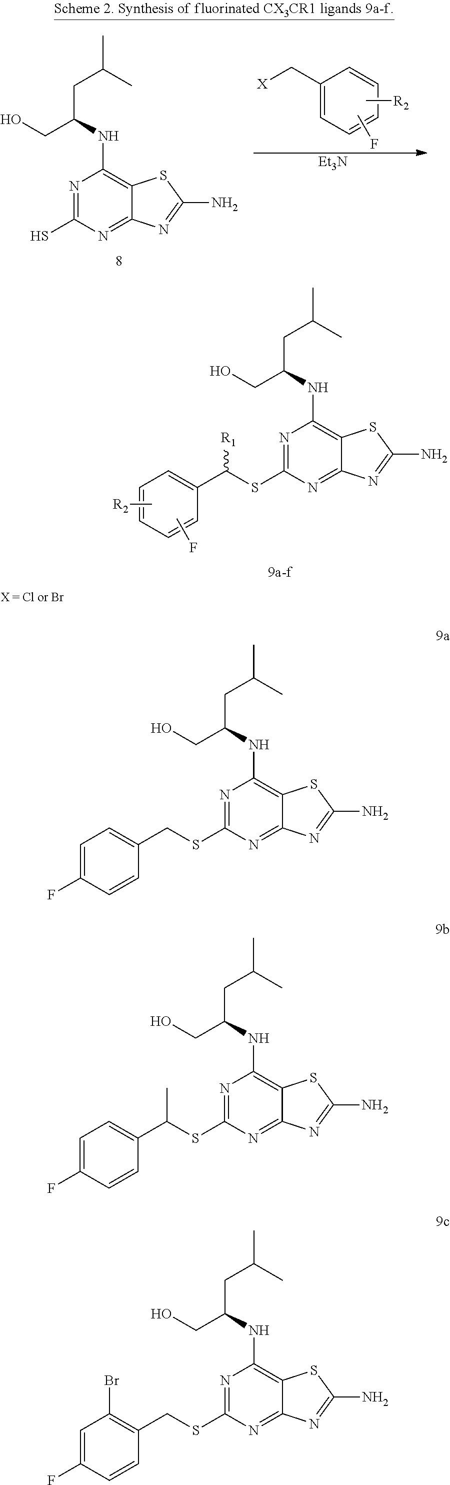

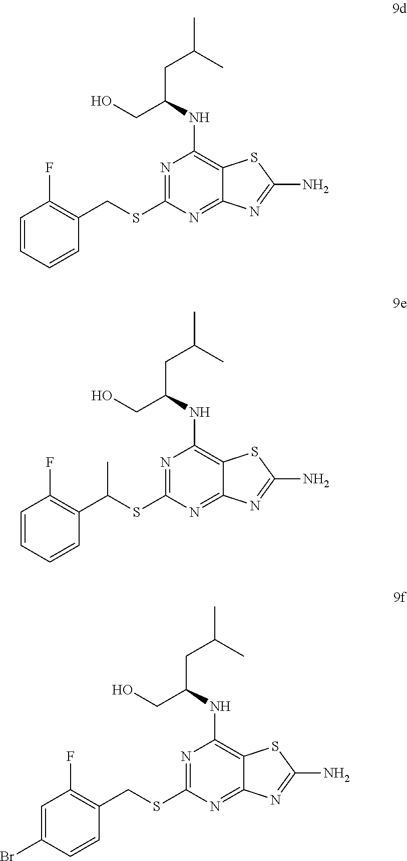 [SMILES: CC(C)C[C@H](CO)Nc1nc(SCc2ccccc2F)nc2nc(N)sc12 ; CC(C)C[C@H](CO)Nc1nc(SC(C)c2ccccc2F)nc2nc(N)sc12 ; CC(C)C[C@H](CO)Nc1nc(SCc2ccc(Br)cc2F)nc2nc(N)sc12]

In principle, another three series of fluorinated 7-amino-5-thio-thiazolo[4,5-d]pyrimidines could be easily accessed starting from general structure A discussed above, as shown in Scheme 3.

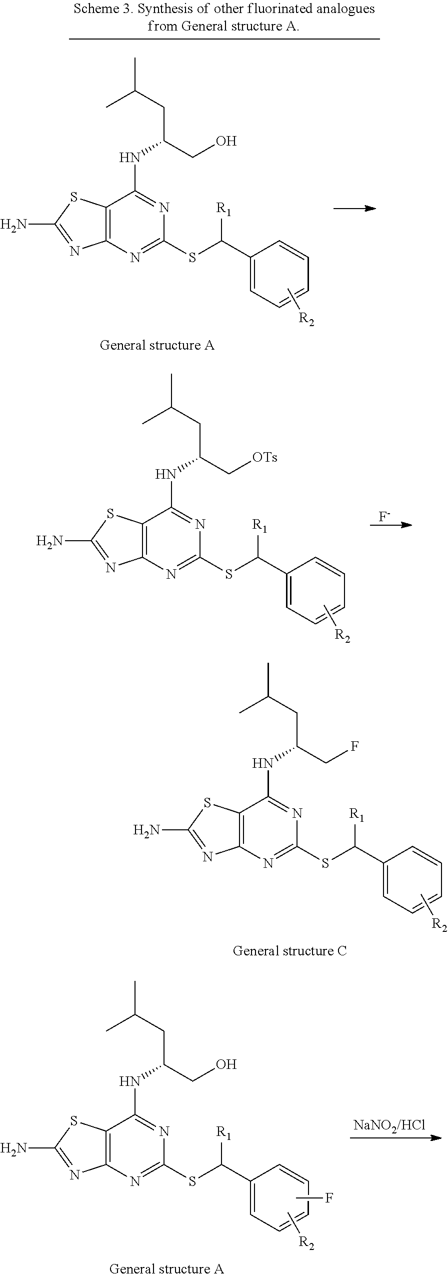

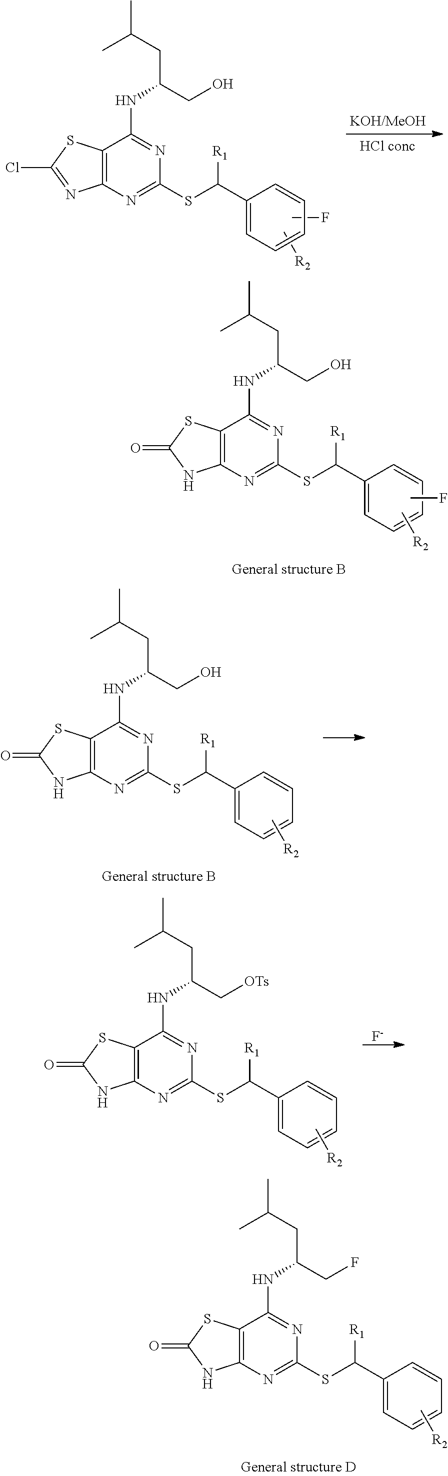

### General Procedure for Synthesis of 9a-f

60 mg (0.2 mmol) of (R)-2-((2-amino-5-mercaptothiazolo[4,5-d]pyrimidin-7-yl)amino)-4-methylpentan-1-ol (8) was dissolved in 1 mL of dry DMSO. To the solution, were added 40 μL (0.29 mmol) of triethylamine and 0.2 mmol of substituted benzylhalide. The reaction was kept overnight. After the DMSO was removed under vacuum, the products 9a-f were purified with flash column chromatography.

### Synthesis of (R)-2-((2-amino-5-((4-fluorobenzyl)thio)thiazolo[4,5-d]pyrimidin-7-yl)amino)-4-methylpentan-1-ol

9a, was obtained in 73% yield. ¹H NMR (400 MHz, DMSO d6): δ 8.01 (br, 2H), 7.45 (m, 2H), 7.12 (m, 2H), 6.92 (d, J=8.1 Hz, 1H), 4.68 (m, 1H), 4.35-4.22 (m, 3H), 3.48-3.40 (m, 1H), 1.68-1.55 (m, 1H), 1.52-1.36 (m, 2H), 1.32-1.15 (m, 2H), 0.91-0.78 (m, 6H). MS: 408 (M+H+)

### Synthesis of (2R)-2-((2-amino-5-((1-(4-fluorophenyl)ethyl)thio)thiazolo[4,5-d]pyrimidin-7-yl)amino)-4-methylpentan-1-ol

9b, was obtained in 80% yield. ¹H NMR (400 MHz, DMSO d6): δ 8.00 (br, 2H), 7.49 (m, 2H), 7.12 (m, 2H), 6.90 (d, J=8.1 Hz, 1H), 4.99-4.92 (m, 1H), 4.75-4.65 (m, 1H), 4.34-4.12 (m, 1H), 3.48-3.40 (m, 1H), 1.68-1.55 (m, 4H), 1.50-1.38 (m, 2H), 0.91-0.78 (m, 6H). MS: 422 (M+H+)

### Synthesis of (R)-2-((2-amino-5-((2-bromo-4-fluorobenzyl)thio)thiazolo[4,5-d]pyrimidin-7-yl)amino)-4-methylpentan-1-ol

9c, was obtained in 69% yield. ¹H NMR (400 MHz, DMSO d6): δ 8.01 (br, 2H), 7.71-7.65 (m, 1H), 7.60-7.58 (m, 1H), 7.23-7.17 (m, 1H), 6.95 (d, J=8.1 Hz, 1H), 4.69 (m, 1H), 4.41 (s, 2H), 4.28-4.18 (m, 1H), 3.48-3.40 (m, 1H), 1.68-1.55 (m, 1H), 1.50-1.36 (m, 2H), 0.91-0.78 (m, 6H). MS: 486 (M+H+)

### Synthesis of (R)-2-((2-amino-5-((2-fluorobenzyl)thio)thiazolo[4,5-d]pyrimidin-7-yl)amino)-4-methylpentan-1-ol

9d, was obtained in 75% yield. ¹H NMR (400 MHz, DMSO d6): δ 8.06 (br, 2H), 7.57-7.51 (m, 1H), 7.35-7.26 (m, 1H), 7.21-7.16 (m, 2H) 7.02 (br, 1H), 4.35 (s, 2H), 4.31-4.21 (m, 1H), 3.48-3.40 (m, 1H), 1.65-1.56 (m, 1H), 1.50-1.38 (m, 2H), 0.90-0.78 (m, 6H). MS: 408 (M+H+)

### Synthesis of (2R)-2-((2-amino-5-((1-(2-fluorophenyl)ethyl)thio)thiazolo[4,5-d]pyrimidin-7-yl)amino)-4-methylpentan-1-ol

9e was obtained in 64% yield, MS: 422 (M+H+) (R)-2-((2-amino-5-((4-bromo-2-fluorobenzyl)thio)thiazolo[4,5-d]pyrimidin-7-yl)amino)-4-methylpentan-1-ol. 9f was obtained in 70% yield. ¹H NMR (400 MHz, DMSO d6): δ 8.11 (br, 2H), 7.57-7.49 (m, 2H), 7.35 (m, 1H), 7.13 (br, 1H), 4.33 (s, 2H), 4.24 (m, 1H), 3.48-3.40 (m, 1H), 1.63-1.55 (m, 1H), 1.48-1.36 (m, 2H), 0.91-0.78 (m, 6H). MS: 486 (M+H+).

### Radiosynthesis of 2-[⁸F]FBTTP and [¹⁸F]9f

The synthesis of 2-[¹⁸F]fluorobenzyl bromide from 2-formyl-N,N,N-trimethylbenzenaminiumin triflate follows the same radiosynthesis strategy previously used to prepare 4-[¹⁸F]fluorobenzyl bromide from 4-formyl-N,N,N-trimethylbenzenaminiumin triflate 3 followed by conjugation to 8 (scheme 4).

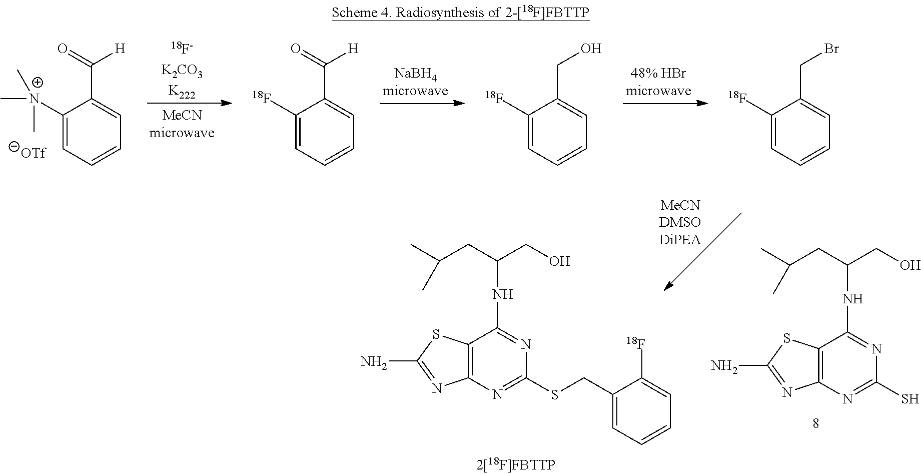 [SMILES: C[N+](C)(C)c1ccccc1C=O ; O=Cc1ccccc1[18F] ; OCc1ccccc1[18F] ; [18F]c1ccccc1CBr ; CC(C)CC(CO)Nc1nc(SCc2ccccc2[18F])nc2nc(N)sc12 ; CC(C)CC(CO)Nc1nc(S)nc2nc(N)sc12]

¹⁸F fluoride was produced by a General Electric PET trace biomedical cyclotron (GE HealthCare) using 18 MeV proton bombardment on an ¹⁸O—H₂O target and trapped on a Chromafix 30-PS—HCO₃ QMA cartridge. The cartridge was eluted with 0.4 mL of a solution of potassium carbonate (3.6 mg/0.4 mL water) into a 3 mL Wheaton reaction vial (159 mCi). To this was added 14 mg 4,7,13,16,21,24-hexaoxa-1,10-diazabicyclo[8.8.8]hexacosane (K2.2.2) in 0.5 mL of acetonitrile and heated to 90° C. under a stream of Argon gas to dryness. Further drying was accomplished by azeotropic distillation using 3×0.5 mL additions of acetonitrile under a stream of Argon gas. The vial was cooled to room temperature and a solution of 14 mg 2-formyl-N,N,N-trimethylbenzenaminiumin triflate in 400 μL acetonitrile was added. The vial is capped and heated at 30 W for 30 sec with the temperature maximum set at 45° C. Next a solution of 1.2 mg sodium borohydride in 200 μL water was added to the Wheaton vial. The vial was capped and heated at 50 W for 30 sec. To the Wheaton vial was then added 800 μL 48 wt % HBr, the vial was capped and heated at 45 W for 300 sec. The crude reaction was then loaded on to an activated (20 mL acetonitrile followed by 20 mL water) Oasis HLB Sep-Pak and washed with 2 mL water. Argon was passed through the Sep-Pak for 30 sec, followed by elution with 1 mL acetonitrile into another 3 mL Wheaton vial. Into this Wheaton vial was then added 20 μL diisopropylethylamine and 2 mg of 2-((2-amino-5-mercaptothiazolo[4,5-d]pyrimidin-7-yl)amino)-4-methylpentan-1-ol 8 dissolved in 200 μL DMSO. This was heated at 80° C. for 10 min, cooled to room temperature and acidified with 50 μL TFA. This was diluted with 3 mL 2/1 water/acetonitrile and injected onto a semi-preparative HPLC (10×250 mm Phenomenex Luna C18 10 micron column, 65/35/0.1 water/acetonitrile/TFA, 4 mL/min). The retention time of 2-((2-amino-5-((2-[18F]fluorobenzyl)thio)thiazolo[4,5-d]pyrimidin-7-yl)amino)-4-methylpentan-1-ol, wt % was 18 min). Yield: 4.8% non-decay corrected (7.65 mCi), 10.2% decay corrected, Specific activity 1500 Ci/mmole, Synthesis time 115 min.

A potential radiosynthesis of (R)-2-((2-amino-5-((4-bromo-2-[¹⁸F]fluorobenzyl)thio)thiazolo[4,5-d]pyrimidin-7-yl)amino)-4-methylpentan-1-ol, [¹⁸F]9f is shown in scheme 5. The preparation of 2-[¹⁸F]fluoro-4-bromobenzaldehyde from 2-nitro-4-bromobenzaldehyde was previously reported (Provisional Patent Application No. 62/067,185). Reduction of the aldehyde followed by conversion to the bromide and conjugation to 8 will give [¹⁸F]9f.

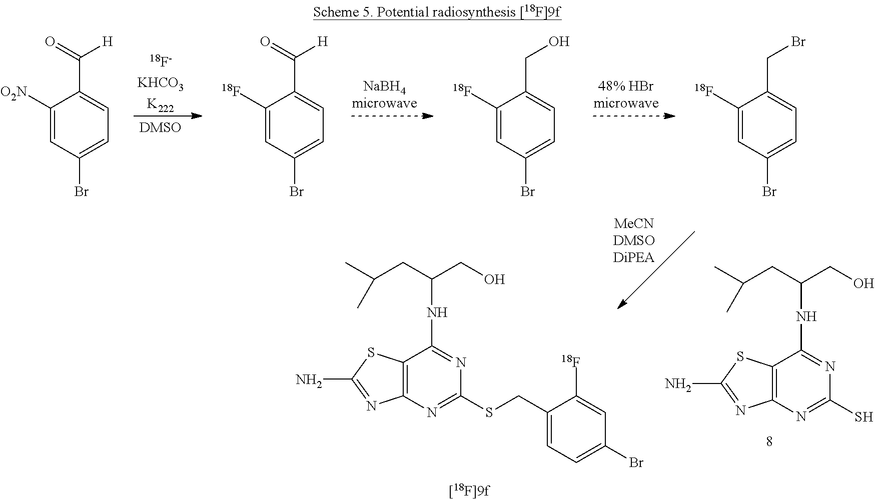 [SMILES: O=Cc1ccc(Br)cc1[N+](=O)[O-] ; O=Cc1ccc(Br)cc1[18F] ; OCc1ccc(Br)cc1[18F] ; [18F]c1cc(Br)ccc1CBr ; CC(C)CC(CO)Nc1nc(SCc2ccc(Br)cc2[18F])nc2nc(N)sc12 ; CC(C)CC(CO)Nc1nc(S)nc2nc(N)sc12]

### Results and Discussion

Imaging of [¹⁸F]9d or 2-[18F]FBTTP in Healthy Mouse.

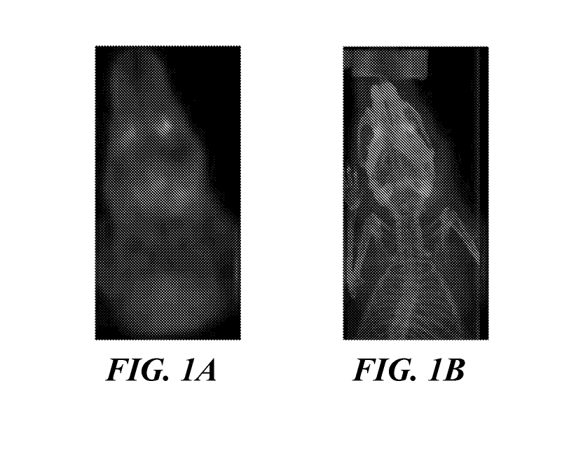

To evaluate in vivo pharmacokinetics of 2-[¹⁸F]FBTTP, 350 μCi of the radiotracer was formulated in 200 μL of saline with 10% ethanol and injected into healthy mice via the tail vein. The images were collected at 10 min, 30 min, 1 hour and 2 hours post-injection. The imaging result at 1 hour time point is shown in FIG. 1.

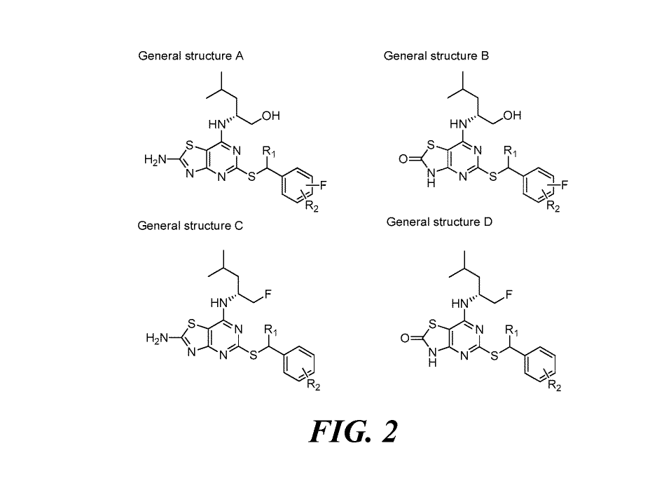

In summary, seven fluorinated 7-Amino-5-thio-thiazolo[4,5-d]pyrimidines that have high binding affinity and specificity to CX₃CR1 has been prepared, and will serve as templates and standards for PET radiotracers. One compound (2-[¹⁸F]FBTTP or [¹⁸F]9d) radiolabeled with the positron emitting radionuclide F-18 has been synthetized in modest radiochemical yield and has demonstrated specific activity favorable in vivo pharmacokinetics in healthy mice. In addition, the chemistry method and radiofluorination method can be adapted for the radiolabeling of other potent fluorinated 7-Amino-5-thio-thiazolo[4,5-d]pyrimidines as listed in FIG. 2. These CX₃CR1 specific low-molecular-weight (LMV) radiotracers are expected to have broad utility, especially for brain imaging, and provide unprecedented and specific information for the detection and treatment of inflammatory diseases.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Cho S Y, Gage K L, Mease R C et al. Biodistribution, tumor detection, and radiation dosimetry of 18F-DCFBC, a low molecular-weight inhibitor of prostate-specific membrane antigen, in patients with metastatic prostate cancer. J. Nucl. Med. 53:1883-1891, 2012.
Hellwig S, Heinrich A, Biber K. The brain's best friend: microglial neurotoxicity revisited. Front. Cell. Neurosci. 2013; (7): 1-11.
Karlstrom S, Nordvall G, Sohn D, Hettman A, Turek D, Ahlin K, Kers A, Claesson M, Slivo C, Lo-Alfredsson Y, Petersson C, Bessidskaia G, Svensson P H, Rein T, Jerning E, Malmberg A, Ahlgen C, Ray C, Vares L, Ivanov V, and Johansson R. Substituted 7-Amino-5-thio-thiazolo[4,5-d]pyrimidines as potent and selective antagonists of the fractalkine receptor (CX₃CR1). J. Med. Chem. 2013; (56): 3177-3190.
Malmquist J, Strom P. Multiple labeling of a potent CX₃CR1 antagonist for the treatment of multiple sclerosis. J. Label. Compd Radiopharm. 2012; (55): 387-392.
Ravert H, Holt D, Dannals R. A microwave radiosynthesis of the 4-[18F]-fluorobenzyltriphenylphosphonium ion. J. Label. Compd. Radiopharm. DOI:10.1002/jlcr.3241, 2014.
Provisional Patent Application No. 62/067,185 to M. G. Pomper, R. C. Mease, and X. Yang for “Scaffolds and multifunctional intermediates for imaging PSMA”.
International PCT Patent Application No. PCT/SE2006/000398 to Nordvall G, Ray C, Rein T, Sohn D. for Novel 5,7-disubstituted [1,3]thiazolo[4,5-D]pyrimidin-2(3H)-one Derivatives published 2006 Oct. 12 (WO 2006/107257 A1).
International PCT Patent Application No. PCT/SE2006/000399 to Nordvall G, Ray C, Rein T, Sohn D. for Novel 5,7-disubstituted [1,3]thiazolo[4,5-D]pyrimidin-2(3H)-one Derivatives published 2006 Oct. 16 (WO 2006/107258 A1).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (Ia) or formula (Ib): 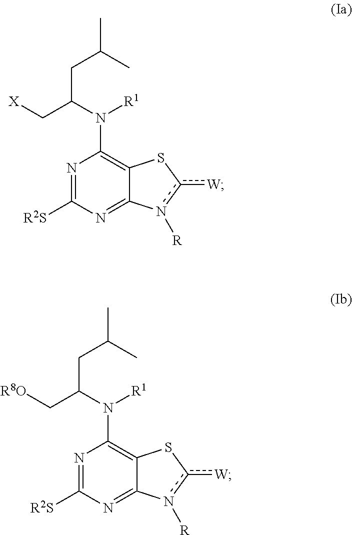
wherein:
R can be present or absent and is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl;
R¹ is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl;
R² is selected from the group consisting of of hydrogen, substituted or unsubstituted alkyl, substituted or unsubstituted aryl, substituted or unsubstituted heteroalkyl, substituted or unsubstituted heteroaryl, substituted or unsubstituted arylalkyl, substituted or unsubstituted alkylaryl, substituted or unsubstituted naphthyl, substituted or unsubstituted biphenyl, and 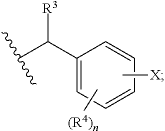
R³ is selected from the group consisting of hydrogen, amine, hydroxyl, carboxyl, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, and substituted or unsubstituted heteroalkylaryl, substituted or unsubstituted naphthyl, and substituted or unsubstituted biphenyl;
R⁴ is selected from the group consisting of halogen, alkoxyl, alkyl, alkenyl, alkynyl, aryl, alkylaryl, arylalkyl, —CN, —CF₃, —CONR⁵R⁶, —SO₂R;
each R⁵ and R⁶ is independently selected from the group consisting of hydrogen, substituted or unsubstituted alkyl, substituted and unsubstituted aryl, and substituted and unsubstituted heteroaryl;
R⁷ is selected from the group consisting of hydrogen, alkyl, hydroxyl, —NR⁵R⁶;
R⁸ is selected from the group consisting of hydrogen and a sulfonyl group;
n is an integer selected from the group consisting of 0, 1, 2, 3, and 4;
X is —NR₅R₆ or is selected from the group consisting of F, Br, and I, and radioisotopes thereof; [symbol]
represents a single or a double bond;
W is selected from the group consisting of ═O, and —NR⁵R⁶;
and stereoisomers or pharmaceutically acceptable salt thereof.

2. The compound of claim 1, wherein the compound of formula (Ia) or (Ib) is selected from the group consisting of: 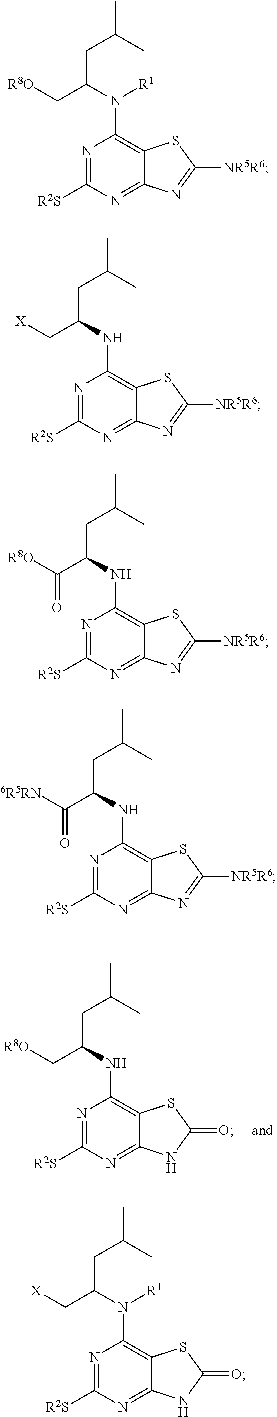
and stereoisomers or pharmaceutically acceptable salt thereof.

3. The compound of claim 1, wherein the compound of formula (Ib) is:

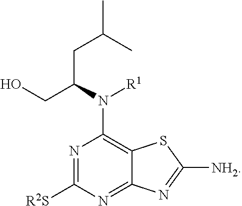

4. The compound of claim 1, wherein the compound of formula (Ib) is selected from the group consisting of:

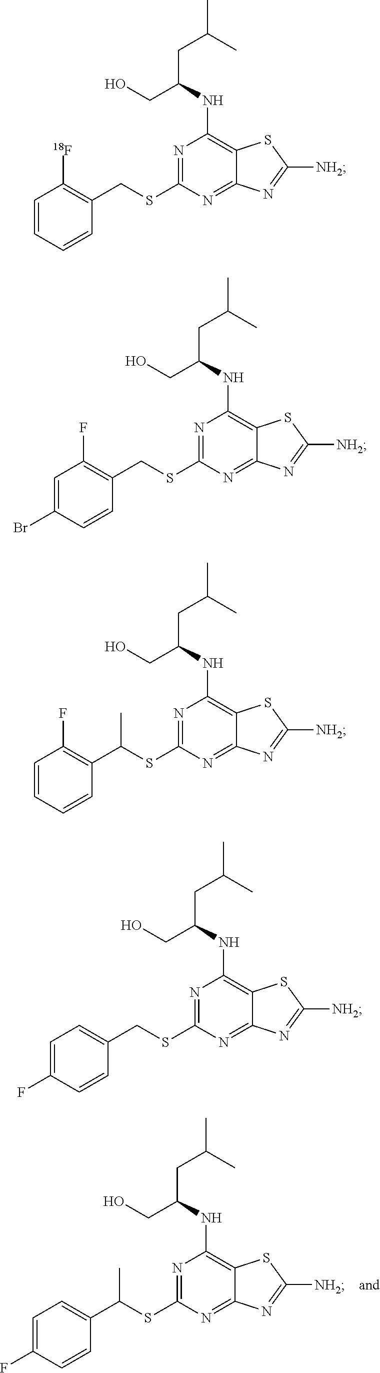 [SMILES: CC(C)C[C@H](CO)Nc1nc(SCc2ccccc2[18F])nc2nc(N)sc12 ; CC(C)C[C@H](CO)Nc1nc(SCc2ccc(Br)cc2F)nc2nc(N)sc12 ; CC(C)C[C@H](CO)Nc1nc(SC(C)c2ccccc2F)nc2nc(N)sc12 ; CC(C)C[C@H](CO)Nc1nc(SCc2ccc(F)cc2)nc2nc(N)sc12 ; CC(C)C[C@H](CO)Nc1nc(SC(C)c2ccc(F)cc2)nc2nc(N)sc12 ; "and"]

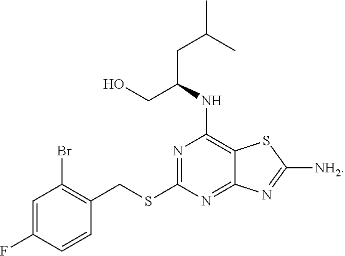 [SMILES: CC(C)C[C@H](CO)Nc1nc(SCc2ccc(F)cc2Br)nc2nc(N)sc12]

5. The compound of claim 1, wherein the compound of formula (Ia) or (Ib) comprises a radioactive isotope suitable for imaging.

6. The compound of claim 5, wherein the radioactive isotope suitable for imaging is selected from the group consisting of ¹⁸F.

7. A method for imaging one or more CX₃CR1-expressing tumors or cells, the method comprising contacting the one or more tumors or cells with an effective amount of a compound of formula (Ia) or (Ib), and making an image, the compound of formula (Ia) or (Ib) comprising: 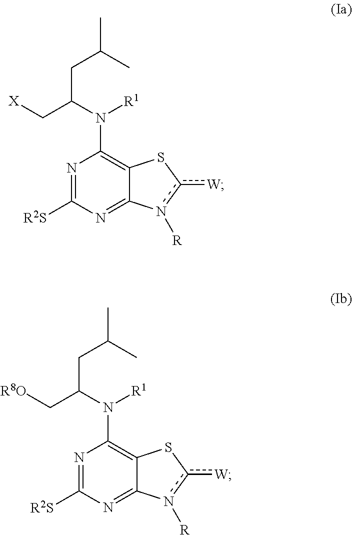
wherein:
R can be present or absent and is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl;
R¹ is selected from the group consisting of hydrogen, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, substituted or unsubstituted heteroalkylaryl, and substituted or unsubstituted naphthyl, substituted or unsubstituted biphenyl;
R² is selected from the group consisting of of hydrogen, substituted or unsubstituted alkyl, substituted or unsubstituted aryl, substituted or unsubstituted heteroalkyl, substituted or unsubstituted heteroaryl, substituted or unsubstituted arylalkyl, substituted or unsubstituted alkylaryl, substituted or unsubstituted naphthyl, and substituted or unsubstituted biphenyl, and 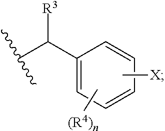
R³ is selected from the group consisting of hydrogen, amine, hydroxyl, carboxyl, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted alkylaryl substituted or unsubstituted arylalkyl, substituted or unsubstituted alkylheteroaryl, and substituted or unsubstituted heteroalkylaryl, substituted or unsubstituted naphthyl, and substituted or unsubstituted biphenyl;
R⁴ is selected from the group consisting of halogen, alkoxyl, alkyl, alkenyl, alkynyl, aryl, alkylaryl, arylalkyl, —CN, —CF₃, —CONR⁵R⁶, —SO₂R⁷;
each R⁵ and R⁶ is independently selected from the group consisting of hydrogen, substituted or unsubstituted alkyl, substituted and unsubstituted aryl, and substituted and unsubstituted heteroaryl;
R⁷ is selected from the group consisting of hydrogen, alkyl, hydroxyl, —NR⁵R⁶;
R⁸ is selected from the group consisting of hydrogen and a sulfonyl group;
n is an integer selected from the group consisting of 0, 1, 2, 3, and 4;
X is ¹⁸F; [symbol]
represents a single or a double bond;
W is selected from the group consisting of ═O, and —NR⁵R⁶;
and radioisotope suitable for imaging;
and stereoisomers or pharmaceutically acceptable salt thereof.

8. The method of claim 7, wherein the compound of formula (Ia) or (Ib) is selected from the group consisting of: 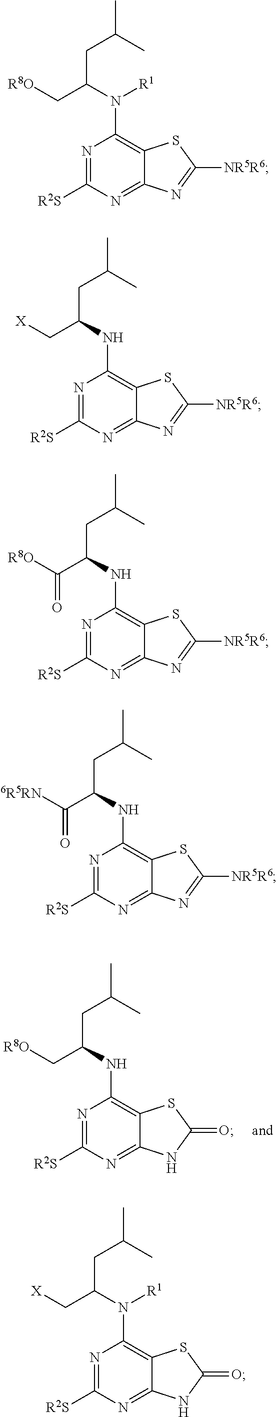
and stereoisomers or pharmaceutically acceptable salt thereof.

9. The method of claim 7, wherein the compound of formula (Ib) is:

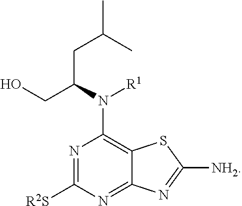

10. The method of claim 7, wherein the compound of formula (Ib) is selected from the group consisting of:

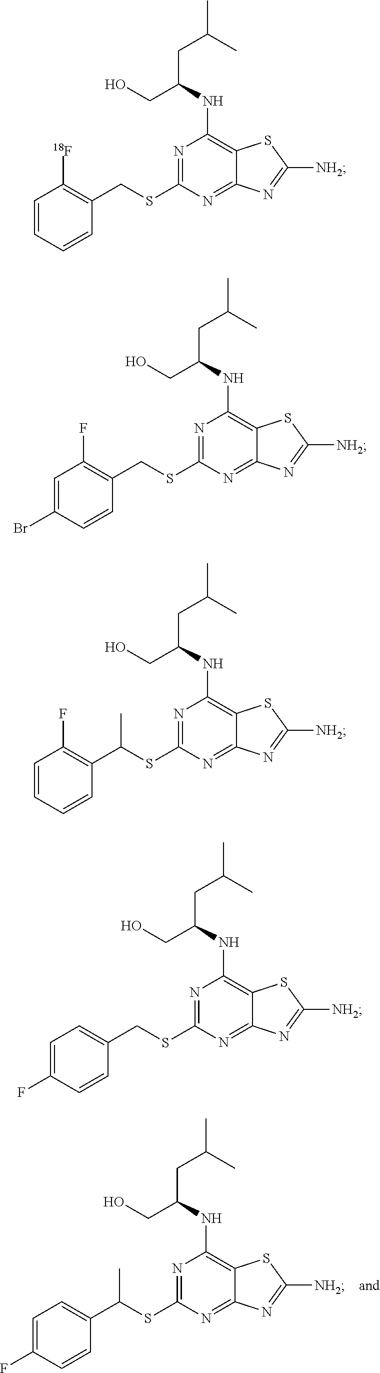 [SMILES: CC(C)C[C@H](CO)Nc1nc(SCc2ccccc2[18F])nc2nc(N)sc12 ; CC(C)C[C@H](CO)Nc1nc(SCc2ccc(Br)cc2F)nc2nc(N)sc12 ; CC(C)C[C@H](CO)Nc1nc(SC(C)c2ccccc2F)nc2nc(N)sc12 ; CC(C)C[C@H](CO)Nc1nc(SCc2ccc(F)cc2)nc2nc(N)sc12 ; CC(C)C[C@H](CO)Nc1nc(SC(C)c2ccc(F)cc2)nc2nc(N)sc12 ; "and"]

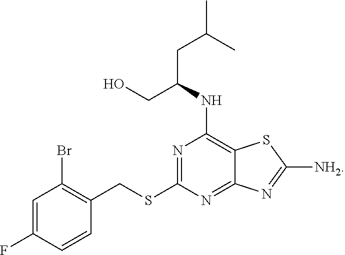 [SMILES: CC(C)C[C@H](CO)Nc1nc(SCc2ccc(F)cc2Br)nc2nc(N)sc12]

11. The method of claim 7, wherein the image is obtained by using positron emission tomography.

12. The method of claim 7, wherein the one or more CX₃CR1-expressing tumors or cells is in vitro, in vivo, or ex vivo.

13. The method of claim 7, wherein the one or more CX₃CR1-expressing tumors or cells is present in a subject.

14. The method of claim 13, wherein the method is non-invasive.

15. The method of claim 13, wherein the compound of formula (Ia) or (Ib) comprising the imaging agent substantially localizes to the tumor or cell within about 60 minutes of administration.
